SEQUENCE LISTING (24 sequences)

SEQ ID NO: 1 - DNA, 20 bp, Unknown
  16S primer - 27f
agagtttgatymtggctcag

SEQ ID NO: 2 - DNA, 19 bp, Unknown
  16S primer -1492r
ggttaccttgttacgactt

SEQ ID NO: 3 - DNA, 19 bp, Unknown
  16S primer -515f
gtgycagcmgccgcggtaa

SEQ ID NO: 4 - DNA, 20 bp, Unknown
  16S primer -806r
  misc_feature (8)..(8): n is a, c, g, or t
ggactacnvgggtwtctaat

SEQ ID NO: 5 - DNA, 22 bp, Unknown
  primer - ITS_1
cttggtcatttagaggaagtaa

SEQ ID NO: 6 - DNA, 20 bp, Unknown
  primer - ITS_2
gctgcgttcttcatcgatgc

SEQ ID NO: 7 - DNA, 20 bp, Unknown
  primer - ITS_3
gcatcgatgaagaacgcagc

SEQ ID NO: 8 - DNA, 17 bp, Unknown
  primer - LR5
tcctgagggaaacttcg

SEQ ID NO: 9 - DNA, 17 bp, Unknown
  primer - PGK
gtygayttcaaygtycc

SEQ ID NO: 10 - DNA, 20 bp, Unknown
  primer - PGK
acaccdggdggrccgttcca

SEQ ID NO: 11 - DNA, 19 bp, Unknown
  primer - ACT512f
atgtgcaaggccggtttcg

SEQ ID NO: 12 - DNA, 20 bp, Unknown
  primer - ACT783r
tacgagtccttctggcccat

SEQ ID NO: 13 - DNA, 28 bp, Unknown
  primer - fusA-f2
tcgcgttcgttaacaaaatggaccgtat

SEQ ID NO: 14 - DNA, 28 bp, Unknown
  primer - fusA-R2
tcgccagacggcccagagccagacccat

SEQ ID NO: 15 - DNA, 20 bp, Unknown
  primer - RPB1-Af
gartgyccdggdcayttygg

SEQ ID NO: 16 - DNA, 23 bp, Unknown
  primer - RPB1-Cr
  misc_feature (3)..(3): n is a, c, g, or t
  misc_feature (9)..(9): n is a, c, g, or t
ccngcdatntcrttrtccatrta

SEQ ID NO: 17 - DNA, 17 bp, Unknown
  primer - LR0R
acccgctgaacttaagc

SEQ ID NO: 18 - DNA, 23 bp, Unknown
  primer - bRPB2-7.1R
cccatrgcytgyttmcccatdgc

SEQ ID NO: 19 - DNA, 20 bp, Unknown
  primer - fRPB2-5F
gaygaymgwgatcayttygg

SEQ ID NO: 20 - DNA, 19 bp, Unknown
  primer - NS1
gtagtcatatgcttgtctc

SEQ ID NO: 21 - DNA, 20 bp, Unknown
  primer - NS4
cttccgtcaattcctttaag

SEQ ID NO: 22 - DNA, 21 bp, Unknown
  primer - SR1R
tacctggttgattctgccagt

SEQ ID NO: 23 - DNA, 23 bp, Unknown
  primer - Btub2Fd
gtbcacctycaraccggycartg

SEQ ID NO: 24 - DNA, 26 bp, Unknown
  primer - Btub4Rd
ccrgaytgrccraaracraagttgtc

(19) United States
(12) Patent Application Publication
(10) Pub. No.: US 2019/0130999 A1
(43) Pub. Date: May 2, 2019

(54) Latent Representations of Phylogeny to Predict Organism Phenotype

(71) Applicant: INDIGO AG, INC., Boston, MA (US)

(72) Inventors: Jacob N. Oppenheim, Boston, MA (US); Tristan Bepler, Cambridge, MA (US)

(21) Appl. No.: 16/170,993

(22) Filed: Oct. 25, 2018

Related U.S. Application Data
(60) Provisional application No. 62/577,670, filed on Oct. 26, 2017.
(60) Provisional application No. 62/639,877, filed on Mar. 7, 2018.

(51) Int. Cl. G16B 40/00; G06F 16/35; C12Q 1/6895; G06N 5/02; G16B 5/00; G16B 30/00; G16B 20/00; G16B 45/00

(52) U.S. Cl. CPC G16B 40/00; G06F 16/35; C12Q 1/6895; G16B 45/00; G16B 5/00; G16B 30/00; G16B 20/00; G06N 5/025

(57) ABSTRACT

Genetic sequence information representative of a first set of organisms is accessed. The first set of organisms can include organisms that include an organism feature and organisms that do not include the organism feature. A latent space representation of k-mers within the first genetic sequence information is generated, for instance using a generative topic model. A generative interpolation model is generated using the latent space representation. The generative interpolation model is configured to classify genetic sequence information representation of a target organism to determine whether the target organism includes the organism feature. Second genetic sequence information representation of each of a second set of organisms is accessed. The generative interpolation model is then applied to the second genetic sequence information to identify which of the second set of organisms are likely to include the organism feature.

### CROSS REFERENCE TO RELATED APPLICATIONS

This application claims the benefit of U.S. Provisional Application No. 62/577,670, filed Oct. 26, 2017 and U.S. Provisional Application No. 62/639,877, filed Mar. 7, 2018, both of which are incorporated by reference in their entirety. This application is related to U.S. application Ser. No. 16/057,387, filed on Aug. 7, 2018, the contents of which are incorporated by reference in their entirety.

### SEQUENCE LISTING

The instant application contains a Sequence Listing in the ASCII format which has been submitted via EFS-Web and is hereby incorporated by reference in its entirety. This ASCII copy, created on Oct. 25, 2018, is named “10104PCTWO1_ST25.txt”, and is 4,770 bytes in size.

### BACKGROUND

Studying the earth's incredible biological diversity is often undertaken from a taxonomic approach. While there is value to such approaches, applied taxonomy has limitations. This is particularly true with regard to certain populations of organisms. Further, when one of the goals is to identify characteristics of organisms that can improve human life, other technical limitations arise. For example, if the goal is to find analogues of Digitalis purpurea (foxglove) to identify other plants or animals that produce similar biochemical compounds, taxonomy is of limited value. This follows, in part, because finding organisms that have been classified and characterized based on special characteristics is rare. Further, some organisms will remain unclassified while having valuable characteristics. Identifying such organisms remains a worthwhile endeavor in spite of the lack of a technical platform or system to perform such analysis.

Valuable products and insights can be obtained, if other approaches can be developed to analyze the biological diversity or subsets thereof. Genetically sequenced organisms that have been cataloged in terms of features are also rare. An organism selected at random is likely to be distinct at levels as broad as the phylum from any other sequenced organism, (e.g., pine tree versus watermelon) especially one with a well annotated genome. This makes understanding the functions of newly sequenced genomes difficult.

From a technical standpoint, the problem is complex. It is likely that only about 20-40% of the genes in a randomly selected strain could be understood functionally through typical bioinformatic approaches. Identifying organisms that share similar phenotypes that have helpful real-world applications remains an unsolved problem or to the extent discoveries are made, traditional methods are time intensive, unpredictable, and not generally reproducible.

The present disclosure provides systems and methods that solve the foregoing problems and others as described herein.

### SUMMARY

In part, the disclosure relates systems and methods that solve the technical problem of identifying target organisms that exhibit desirable organism features.

The number of possible target organisms to evaluate to find a given organism feature is vast. Given that it would take a commercially prohibitive time scale to investigate all possible target organisms, the ability to prioritize organisms to investigate can enable an efficient allocation of investigative resources. Accordingly, in part, the solution described herein solves the technical problem of prioritizing target organisms for analysis, testing, and recommendation, possibly resulting in higher performance agriculture, with an increased chance to better address the nutritional needs of a growing worldwide population. In particular, latent spaces as discussed in more detail herein can be used to perform machine learning and/or interpolation of organism features to generate a prioritized list of target organisms, which can, for instance, advantageously expedite the development of beneficial endophyte strains or other beneficial organisms for a variety of purposes.

In part, the solution described herein can apply the technical solution of collecting observable data regarding an organism, which may include direct or indirect measurements of various parameters relating thereto and how such parameters change when subjected to different environmental conditions and stresses. Further, the observable data can include data from the public record, such as third party studies of a particular organism. In part, the solution described herein uses latent variables representative of organisms to identify various phenotypes and features of interest associated with the organisms.

In particular, various embodiments of the solution described herein include the accessing of genetic sequence information representative of a first set of organisms that include a known state of an organism feature (e.g., a known first subset of the first set of organisms includes the organism feature, and a known second subset of the first set of organisms does not include the organism feature). A latent space representation of k-mers within the first genetic sequence information is generated, for instance using a generative topic model. A generative interpolation model is trained using the generated latent space representation. The generative interpolation model is configured to classify genetic sequence information representative of a target organism to determine whether the target organisms includes the organism feature. Second genetic sequence information representative of a second set of organisms is accessed, and a set of target organisms is identified from the second set of organisms by applying the generative interpolation model to the second genetic sequence information, for instance after generating a latent space representation of the second genetic sequence information.

In some embodiments, the generative topic model is a latent Dirichlet allocation model. The generative topic model can be pre-trained, for instance on a set of training genetic sequence information representative of a first set of organisms that include the organism feature and a second set of organisms that do not include the organism feature. The generative topic model can be periodically updated, for instance in response to receiving additional genetic sequence information for inclusion in the set of training genetic sequence information, or in response to receiving feedback indicating that a predictiveness of one or more k-mers for the organism feature is less than a threshold predictiveness.

In some embodiments, the latent space representation is re-generated in response to a triggering event, and the re-generated latent space representation includes at least one input variable not included in the latent space representation. Examples of a triggering event include a passage of a threshold interval of time, determining that a threshold portion of the identified subset of organisms does not include the organism feature, and obtaining additional genetic sequence information for use in generating the latent space representation. The latent space representation can include a matrix, wherein each row of the matrix corresponds to an organism of the first set of organisms, and wherein each column of the matrix corresponds to a presence of one or more k-mers within genetic sequence information. In some embodiments, each row of the matrix corresponds to a community of organisms, for example a community of organisms isolated from a particular environment or individual host organism. The latent space representation can also include a sparse representation of k-mers within the first genetic sequence information.

The genetic sequence information used by the solution described herein can include DNA sequence information (for example, metagenome sequence information, whole genome sequence information, marker gene sequence information, etc.), RNA sequence information (for example, metatranscriptome sequence information, whole transcriptome sequence information, gene transcript sequence information, etc.), and/or amino acid sequence information (for example, metaproteome sequence information, proteome sequence information, the amino acid sequence of a particular gene, etc.). The generative interpolation model trained by the solution described herein can be trained based in part on a distance between input variables in the latent space representation.

Applying a generative interpolation model to genetic sequence information can include converting the genetic sequence information into a latent space representation of the genetic sequence information, and determining a likelihood that each organism associated with the genetic sequence information includes an organism feature based on a covariance between a weighted average value of a variable in the latent space representation and a variable in a latent space representation used to train the generative interpolation model. The generative interpolation model can be a Gaussian process model or any other suitable model or classifier. The generative interpolation model can classify genetic sequence information by determining that the likelihood that a corresponding target organism includes an organism feature corresponding to the generative interpolation model is greater than a predetermined likelihood threshold. Organisms identified as likely to include the organism feature can be prioritized for testing to determine if the organisms include the organism feature. In some embodiments, the organisms identified as likely to include an organism feature can be prioritized for testing for a different or unrelated organism feature or trait. In yet other embodiments, different organisms can be prioritized for testing based on the identified organisms. For instance, the yield and disease resistance of a set of crops, when treated with an identified organism likely to include an organism feature, can be prioritized for testing.

The organisms used to generate the latent space representation and the organisms being classified can include, for instance, bacteria, archaebacteria, eubacteria, protista, fungi, plantae, and animalia. In embodiments where the organisms are plants, the plants can be monocots or dicots, and can include corn, soybeans, cotton, wheat, rice, barley, oats, tomatoes, canola, and sorghum. The set of organisms used to generate the latent space representation can include any number of organisms, for instance 10 or less organisms, 50 organisms, 100 organisms, 500 organisms, 1000 or more organisms, or any number in between. Likewise, the set of organisms used to generate the latent space can include any number of organism communities that each include any number of individuals, and can also include a combination of individual organisms and organism communities.

The organism feature associated with the generative interpolation model can include but is not limited to: a genomic composition, a frequency of biosynthetic gene clusters, a taxonomic categorization, a morphology, an environmental niche, a lifestyle, a resistance to desiccation, a spore formation, a suitability for manufacturing or harvesting, a viability, a compatibility with commercial practices, a stability in viability over time or ranges of environmental conditions, a compatibility with select formulations and chemical preparations, a chemical diversity production, a metabolite production, a pathogenicity, a toxicity, a metagenomic composition, a frequency of genes, a yield associated with an organism, a yield increase associated with an organism, and a crop performance.

In particular embodiments, a generative topic model can be trained on crop sequence information, and a generative interpolation model can be trained with a latent space representation generated using the generative topic model. A requesting entity, such as a crop grower, crop broker, or agronomist, can request a recommendation for a crop with a particular crop feature associated with the generative topic model. The crop feature, or a type of crop used to train the generative topic model, can be selected by the requesting entity. In response, a set of crops likely to include the crop feature can be identified by applying the generative interpolation model to sequence information associated with a set of candidate crops, and a client device interface can be modified to display the identified set of crops. The sequence information associated with a set of candidate crops can be accessed from local memory, from communicatively coupled databases, or from one or more client device associated with the requesting entity. In some embodiments, the identified set of crops are all crops classified by the generative interpolation model that are associated with an above-threshold probability of including the crop feature. The recommendation can be requested via an interface element displayed by the client device.

In some embodiments, a generative topic model can be trained on crop sequence information of particular genotypes of a selected crop, and a generative interpolation model can be trained with a latent space representation generated using the generative topic model. In some embodiments, a requesting entity can request a recommendation for a particular crop with a particular crop feature associated with the generative topic model. For example, the selected crop may be corn and the selected crop feature may be yield under drought conditions. The crop and crop feature used to train the generative topic model, can be selected by the requesting entity. In response, a set of genotypes of a particular crop or combinations of genotypes of a particular crop likely to include the crop feature can be identified using the generative interpolation model, and a client device interface can be modified to display the identified set of genotypes or combinations of endophytes. In some embodiments, the identified set of crop genotypes or combinations thereof are all crop genotypes or combinations thereof classified by the generative interpolation model that are associated with an above-threshold probability of including the crop feature.

The crop feature associated with the generative interpolation model can be an above-threshold expected crop performance, and the displayed identified crops can further include a measure of expected crop performance for each crop determined based on the probability that the crop includes the crop feature. In some embodiments, the latent space representation can be re-generated, for instance based on whether or not the identified crops actually include the crop feature, or availability of the identified crops in a specified geographic area. The generative interpolation model can then be re-trained using the re-generated latent space representation, and a second set of crops that are likely to include the crop feature can be identified using the re-trained generative interpolation model.

In some embodiments, a generative topic model can be trained on microbe sequence information, and a generative interpolation model can be trained with a latent space representation generated using the generative topic model. In some embodiments, a requesting entity can request a recommendation for a particular microbe or community of microbes with a particular organism feature associated with the generative topic model. For example, the selected crop may be soy and the selected organism feature may be resistance to soybean sudden death syndrome (SDS) of a treated plant. The crop and organism feature used to train the generative topic model can be selected by the requesting entity. In response, a set of microbes or communities of microbes likely to include the organism feature (e.g., resistance to SDS) can be identified using the generative interpolation model, and a client device interface can be modified to display the identified set of microbes or communities of microbes. In some embodiments, the identified set of microbes or communities of microbes can be displayed as recommended crop treatment options. For example, an interface displayed by a device of a requesting entity can identify a particular crop, and can display each identified set or community of microbes, along with a description of the organism feature that identified set or community of microbes is likely to include and/or a representation of the likelihood that the set or community of microbes includes the organism feature. In some embodiments, the identified set of microbes or communities thereof are all microbes or communities thereof classified by the generative interpolation model that are associated with an above-threshold probability of including the organism feature.

### BRIEF DESCRIPTION OF THE DRAWINGS

The methods, compositions and apparatus of the present disclosure can be more fully understood in reference to the following detailed description when considered with in connection with the following drawings. The reference numerals included in the Detailed Description herein refer to corresponding reference numbers within the Figures.

FIG. 1A is a schematic diagram of a platform or system suitable for collecting training data and generating target candidate recommendations based on one or more models and an observed feature of interest

FIG. 1B is a schematic diagram of a phylogenetic tree showing differing levels of relatedness and also have one or more features of interest.

FIG. 1C is a schematic diagram of a latent space representation corresponding to a two-dimensional vector space that has been generated with regard to the tree of FIG. 1B.

FIG. 2A shows an illustrative process for prediction of at least one phenotype of an organism and the sequences from which the one or more phenotypes arise in accordance with some embodiments of the present disclosure.

FIG. 2B shows an illustrative process for prediction of the plant growth promotional effect of an organism and the sequences from which the one or more phenotypes arise in accordance with some embodiments of the present disclosure.

FIG. 3 is a plot showing the correlation between topic model distance and alignment distance.

FIG. 4 shows the distribution of the entropies of the k-mer topics, calculated as the entropy of the distribution of k-mer frequencies within each topic.

FIG. 5A is a plot of actual values of IaaH gene clusters versus values predicted with regard to characterized bacterial strains using methods of the disclosure.

FIG. 5B is a plot of actual values of IaaH gene clusters versus values predicted with regard to uncharacterized fungal strains using methods of the disclosure.

### DETAILED DESCRIPTION

### Definitions

As used herein, unless otherwise stated, the singular forms “a,” “an,” and “the” include plural reference. Thus, for example, a reference to “a target candidate” is a reference to one or more target candidates.

As used herein, “about” will be understood by persons of ordinary skill in the art and will vary to some extent depending upon the context in which it is used. If there are uses of the term which are not clear to persons of ordinary skill in the art, given the context in which it is used, the term “about” in reference to quantitative measurements or values will mean up to plus or minus 10% of the enumerated value.

The term “automatic” or “automated” refers to steps or actions performed without direct human interventions, that is, under computer-based software control, robotic control, or the control of other machines, systems, and devices, unless otherwise specified.

The term “classifier” refers to a method or function that categorizes or classifies input data. A given classifier can use one or more parameters such as k-mer frequency, a distance metric, image representations, and other parameters and constraints which are user specified in various embodiments.

A “beneficial” endophyte does not cause disease or harm the host plant otherwise. Endophytes can occupy the intracellular or extracellular spaces of plant tissue, including the leaves, stems, flowers, fruits, seeds, or roots. An endophyte can be, for example, a bacterial or fungal organism, and can confer a beneficial property to the host plant such as an increase in yield, biomass, resistance, or fitness. An endophyte can be a fungus or a bacterium. As used herein, the term “microbe” is sometimes used to describe an endophyte. As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is an endophyte, for example a bacterial or fungal endophyte, which is capable of living within a plant.

As used herein, in reference to a characteristic of an organism or community, the term “feature” is used interchangeably with “phenotype”, “organism feature”, and “biological feature”. It should be noted that although the term “organism feature” may be used herein with reference to a feature of a particular organism, in practice, the use of the term “organism feature” can also refer to a feature of a community of organisms. It may be desirable to predict a broad range of features of an organism or a community of organisms, including but not limited to: genomic composition, transcription of one or more genes, abundance or presence of one or more proteins, abundance or presence of one or more chemical compounds, incidence of disease, susceptibility or resistance to disease or infection, taxonomic categorization, morphology, behavior, environmental niches, lifestyle, resistance to desiccation, spore formation, suitability for manufacturing or harvesting, viability, compatibility with commercial practices, stability in viability over time or ranges of environmental conditions, compatibility with relevant formulations and other chemical preparations, biological diversity production, metabolite production, pathogenicity, toxicity, yield or increased yield, or other features, including the ability to induce or modulate these features in another organism. Any feature of an organism that is assumed to be biologically controlled can be used; these may be referred to interchangeably as “phenotypes”, “organism features”, “features” or “biological features”.

As used herein, a “generative model” refers to a model, such as a machine learning model that is trained using a set of data, which as a result of being trained, can generate new targets that follow the probability distribution of the training set. A generative model can be used to implement an unsupervised learning system. A generative model can generate the observed values used to train it and variables that can be modeled based on their fit to the probability distribution of the training set. One or more generative models can be generated using observed k-mer frequency to understand and model how sequences differ.

As used herein, the term “yield” can refer to a component per unit of measure. For example, a component can be the biomass of a community or an organism, including the biomass of a particular tissue of the organism; an abundance of an organism or an abundance of a particular aspect of a composition or product derived from an organism (for example, a component may be a tissue such as a seed, or an aspect of a composition such as fat, oil, carbohydrate, protein, mineral, or metabolite content of an organism or tissue or product thereof). As non-limiting examples, a unit of measure may be a production area (for example, a field, greenhouse, or fermentation vessel), an individual tissue, an individual organism, a community, or a unit of area or a measure such as an acre, a bushel, or a liter.

As used herein in reference to agricultural plants or features of a given organism, the phrase “increased yield” refers to an increase in biomass or seed weight, dressing weight per organism, seed or fruit size, seed number per plant, seed number per unit area, weight of grain per bushel, bushels per acre, tons per acre, kilo per hectare, carbohydrate yield, cotton yield, product yield (such as an increased oil composition per liter of culture media), and the like. An increased yield can also refer to an increase production of a component of, or product derived from, an organism or tissue or of unit of measure thereof, such as an increased protein yield of a pound of grain or an increased oil yield of a seed. Complex features such as yield and viability are likely to be better estimated by this methodology. An organism's features may be observed under natural or experimental conditions.

A “k-mer” or “kmer” is a subsequence of k contiguous characters within a second sequence, where k is any real integer. k is at least 1 character. In some embodiments, k is greater than 2 and less than 10 characters. For example, a subsequence that is 6 nucleotides in length may be referred to as a 6-mer and a subsequence that is 20 nucleotides in length may be referred to as a 20-mer. k-mer size is chosen to i) maximize the ability to predict/make recommendations when identifying target candidates, while ii) minimizing computational load as the number of nucleotide k-mers scales as 4^{k}. In practice, the smallest k-mer size that accurately represents phylogeny in the training data is chosen.

As used herein, an “organism” may be any functional unit comprising at least one molecule of nucleic acid. An organism may be a eukaryote, prokaryote, or virus. For example, an organism may be a human, plant, or a microorganism. The methods of the present disclosure are also particularly useful for analysis of microbes and microbial communities.

As noted above, the system described herein is applicable to inferring the properties of individual organisms or communities of organisms. “Communities” may be naturally occurring, for example plant communities in a prairie ecosystem or microbiome communities of the human gut. Communities may be artificially constructed, for example: a plurality of organisms comprising one or more members which have been “modified” or a plurality of naturally occurring isolated bacteria and fungi which are combined in a synergistic artificial composition. For example, a synergistic artificial composition may include two or more organisms which are combined by human endeavor in a manner not found in nature. An individual organism or community may have undergone random or directed modification.

In some embodiments, a treatment may include a modified microbe or plant or plant element (which may include a transgene). A microbe or plant or plant element is “modified” when it comprises an artificially introduced genetic or epigenetic modification. The modification may be introduced by a genome engineering or genome editing technology. The genome engineering or editing may utilize non-homologous end joining (NHEJ), homology directed repair (HDR), or combinations thereof, and may be implemented with a Class I or Class II clustered regulatory interspaced short palindromic repeats (CRISPR) system. In various embodiments, the CRISPR system is CRISPR/Cas9, or CRISPR/Cpf1. The genetic or epigenetic modification may be introduced by a targeted nuclease, which may include: transcription activator-like effector nuclease (TALEN), zinc finger nuclease (ZNF), Cas9, Cas9 variants, Cas9 homologs, Cpf1, Cpf1 variants, Cpf1 homologs, and combinations thereof. Likewise, the genetic or epigenetic modification may be introduced by treatment with a DNA methyltransferase inhibitor such as 5-azacytidine, or a histone deacetylase inhibitor such as 2-amino-7-methoxy-3H-phenoxazin-3-one. Finally, the genetic or epigenetic modification may be introduced via tissue culture.

As used herein, a “target candidate”, “candidate target”, or “target organism” is an organism such as, but not limited to an endophyte, that has a beneficial feature or that may be determined to have a beneficial feature after being tested or analyzed once initially identified.

As used herein, “training data” is data that is used to train a model. Training data may be observed data, including any of the data obtained using the systems disclosed herein. Training data may be enriched or labeled by machines or people to ensure it is relevant for the application for which a given model is being trained. The terms “training data” may be used interchangeable with “reference data”, likewise “training dataset” is equivalent to “reference dataset.”

A “sequence” is a character string representation of nucleotides within a polynucleotide, including deoxyribonucleic acid (DNA) or ribonucleic acid (RNA), or amino acids within a polypeptide or protein sequence. A sequence may represent all of the genetic or proteomic information within a single organism or within a community of organisms. Sequences may represent one or more genes, one or more genomic regions, one or more chromosomes or plasmids, or one or more genomes. The sequences may represent all microorganisms in a culture collection, the culture collection containing at least two microorganisms, and generally being a much larger collection.

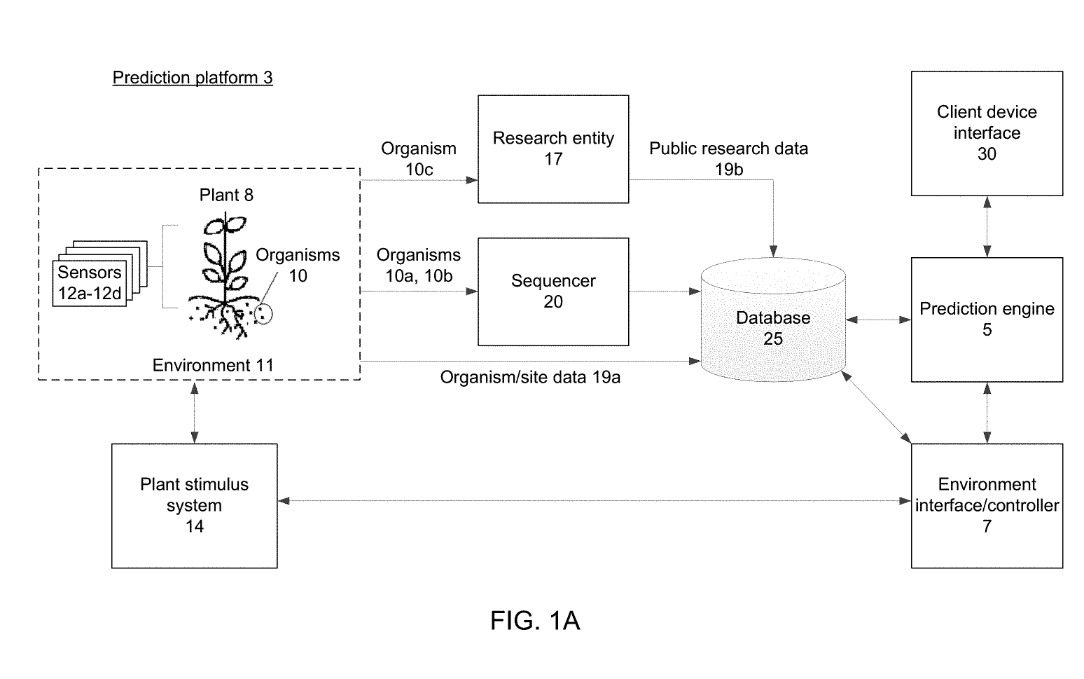

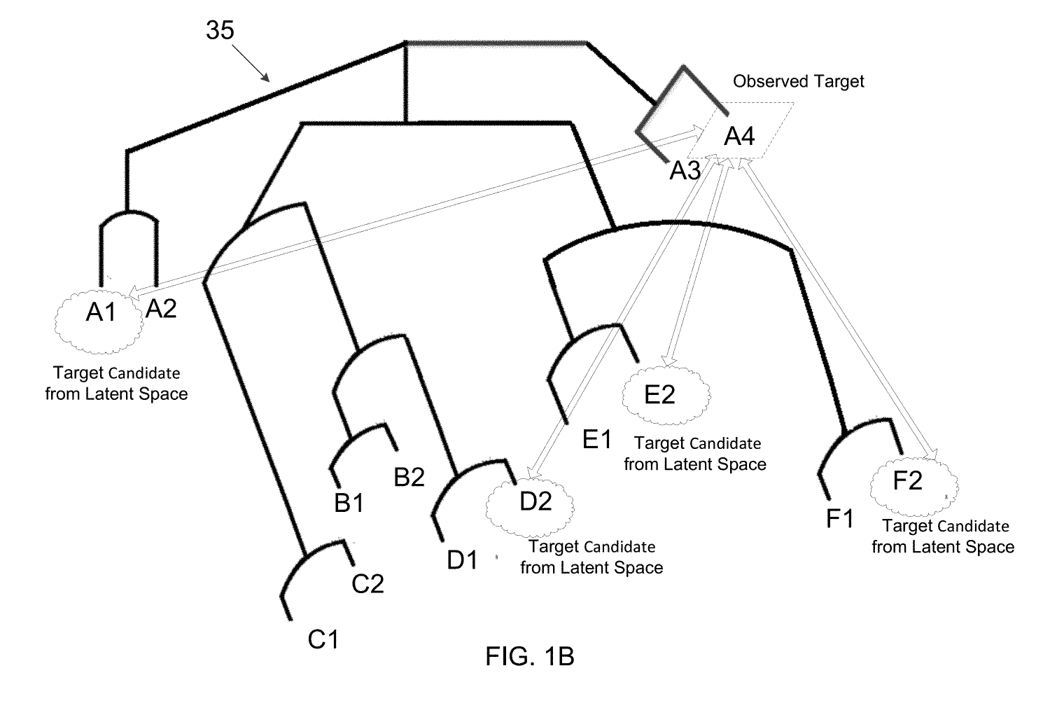

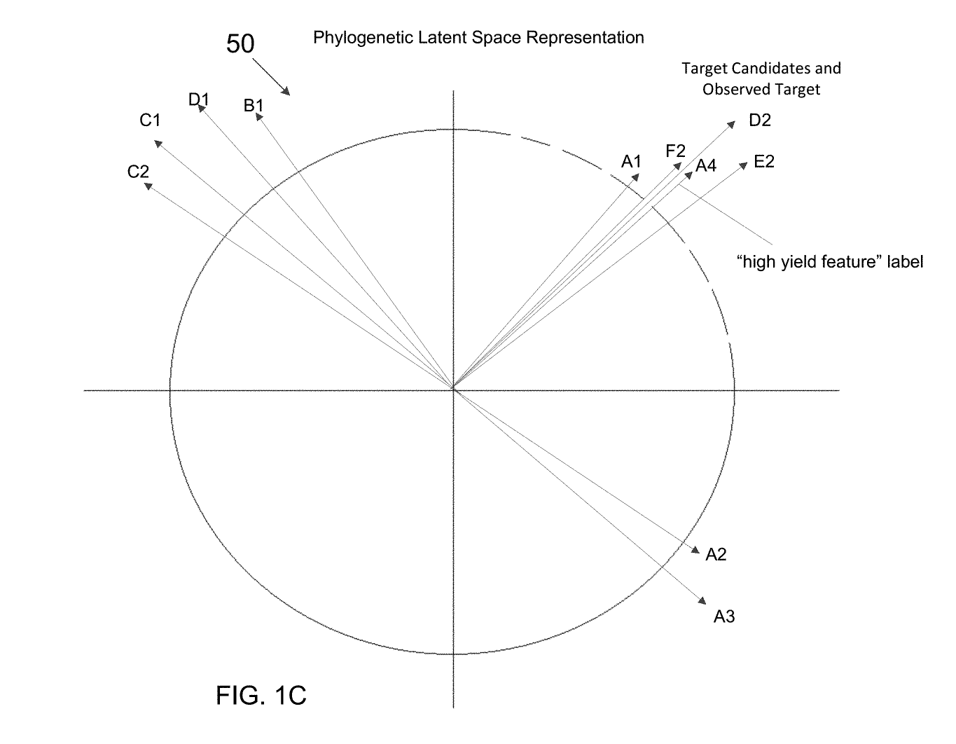

In part, the disclosure applies machine learning, modeling, distance metrics, pattern matching, and other computer-based and data science technologies as part of an integrated platform suitable for solving various technical problems in the field of biology and the subfields of genetics and bioinformatics as described herein. An exemplary system or platform 3 is shown in FIG. 1A that is suitable for performing the methods and operations on data, vector spaces, machine learning software modules, latent space representations, and the other components and tools described and depicted herein.

The platform 3 of FIG. 1A includes a prediction engine 5; an environment interface/controller 7; a plant environment 11 that includes a plant 8, organisms 10, and sensors 12a-12d; a plant stimulus system 14; a research entity 17, a sequencer 20, a database 25, and a client device interface 30. It should be noted that in other embodiments, the platform 3 includes fewer, additional, or different components than those illustrated in FIG. 1A. The prediction engine 5 (or “system 5”, “computer system 5”, “computing device 5” hereinafter) includes one or more computing devices or computer systems. The system 5 includes one or more electronic memory devices and processors and also includes various network communication devices. The system 5 can include one or more components described herein relating to computing, networking, ASICs, and software. The system 5 can receive data from various entities as shown using a data channel or network. The system 5 can be operably connected to or in communication with an environment interface/controller 7. The environment interface/controller 7 includes various components, which can include processors or computing devices. In some embodiments, the computer systems 5 can include a cloud-based server or servers, each with one or more processors, acting in concert to enable the functionalities described herein, for instance by executing instructions stored in one or more non-transitory computer-readable storage mediums.

Still referring to FIG. 1A, the interface and controller can send and receive signals and data to devices at various locations that have plants 8 growing, being tested or being processed to remove organisms 10. The plants 8 are also typically in proximity to organisms 10, which can include any organisms. In one preferred embodiment, the organism 10 is a beneficial endophyte. The 8 are typically in an environment 11 which can be a lab, a farm, a grow room, or other location. In various embodiments, a given environment and the resident plants 8 and organisms 10 are subject to monitoring via various sensors 12a, 12b, 12c, and 12d. The sensors can be any suitable sensor or detecting device. Although four sensors are shown, this is only for the sake of example and any number of sensors can be used. The sensors can include pH, temperature, soil, humidity, pressure, lighting, chemical, air quality, gas detection, oxygen, carbon dioxide, cameras, drones, other transducers, transceivers, receivers, transmitters and combinations of the foregoing. Other sensors suitable for measuring plant, organism, and environmental parameters can be used without limitation. The activation and relaying of data from the sensors 12a, 12b, 12c, and 12d can be performed using the interface/controller 7 or through the computing device 5. The computing device can also run various analytical and predictive tools to process data and store it in a database 25. The data obtained with regard to the plants 8, environment 11 and organisms 10 is referenced as organisms/site data (SOD). This SOD data 19a can include all of the data obtained with regard to environment 11 and its inhabitants or selected subsets thereof.

A given environment 11 can also include a plant stimulus system 14. The plant stimulus system 14 can change various parameters that affect the plant 8 and the organisms 10. The plant stimulus system can include various subsystems and components to spray pesticide, change the temperature, simulate drought, simulate frost, and otherwise replicate any real world conditions relative to the environment 11 in which the plant 8 and the organisms 10 are disposed. These environmental changes can be controlled using interface/controller 7 and the resultant data collected from the plant can be stored in database 25. Although one database is shown, other databases can be used. As data is changed based on findings and the use of machine learning to identify target candidates from the organisms 10, the database and the models used by system 5 can be updated through one or more feedback loops.

In particular, the systems and methods of the present disclosure are directed to the technical problem of determining relatedness with respect to certain phenotypes between known organisms that have been genetically analyzed and phenotypically categorized and unknown and/or uncategorized organisms. For example, as part of a research study, plants, fungi, microorganisms or another organism is analyzed to obtain genetic information about such an organism such as through group 17 or by experiments and plant studies with regard to environment 11.

As shown in FIG. 1A, two representative organisms 10a and 10b, such as endophytes, from the environment 11 are obtained and processed by sequencers 20. In this way, one or more genetic sequences from each organism 10a, 10b that was living with plants 8 can be stored in the database 25. To the extent necessary, the computing device 5 can perform data normalization, filtering, and noise removal with regard to all of the data and signals collected with regard to the plants 8 or from the public research efforts of third parties 17. As disclosed herein, various data and information of interest is collected at various environments 11, such as shown in FIG. 1A, in which plants and other organisms are grown and experimentally tested with regard to various features and parameters. Each environment, which can include a farm, a lab, or other suitable habitat or life support for a given plant or organism, communicates sensor data with a network, such as a LAN, the internet, cellular, or any other any suitable network.

As shown in FIG. 1A, third parties can conduct research with regard to various organism such as organisms 10c, which also may be an endophyte or other organism. This public research data (PRD) 19b, which can also include genetic sequences, functional details relating to phenotype of organism 10c, and other information can be processed and stored in database 25. The information from other researchers 17 that are made available to the public can include information about the genes and phenotypes of other similar organisms.

The system and methods of the present invention provide a solution for overcoming the challenges of functional prediction using traditional bioinformatics methods. Examples of traditional methods include, but are not limited to, PICRUSt (Langille M G I, Zaneveld J, Caporaso J G, et al. Predictive functional profiling of microbial communities using 16S rRNA marker gene sequences. Nature biotechnology. 2013; 31(9):814-821. doi:10.1038/nbt.2676) and PRISM (available at magarveylab.ca/prism). Traditional methods, such as PICRUSt, rely on taxonomic interpolation, using an organism's taxonomy to identify closest relatives and infer its likely properties, including the genes it possesses, the niches it inhabits, or its lifestyle, and assume that organisms within certain clades all share certain properties. These methodologies require expert knowledge to annotate which functions are shared at which taxonomic levels. As a result, these are not suited for automated systems to identify target candidates. Further, the necessity for expert knowledge makes taxonomic-based approaches slow, limited in scope or both. Clearly, such methods are not suitable for real-time analysis or substantially real-time analysis to select target candidates.

Additionally, they do not properly model an organism's phylogeny. Further, such legacy techniques assume Linnean taxonomic accurately reflects phenotypic evolution. Errors can arise from such an approach because it treats all organisms at the same taxonomic rank the same. These methods are limited by their reliance on Linnaean taxonomy or a predefined phylogeny for inferring neighborliness. For example, the seven-level Linnaean hierarchy, created for the study of macrofauna, has little to no meaning in the microbial world.

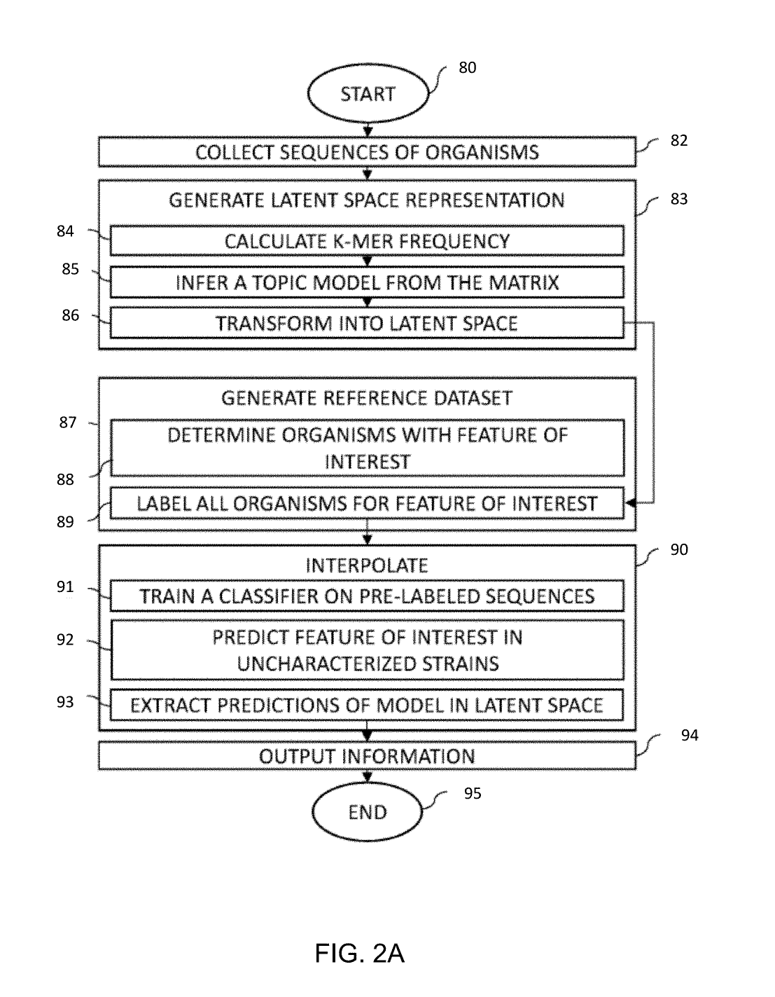

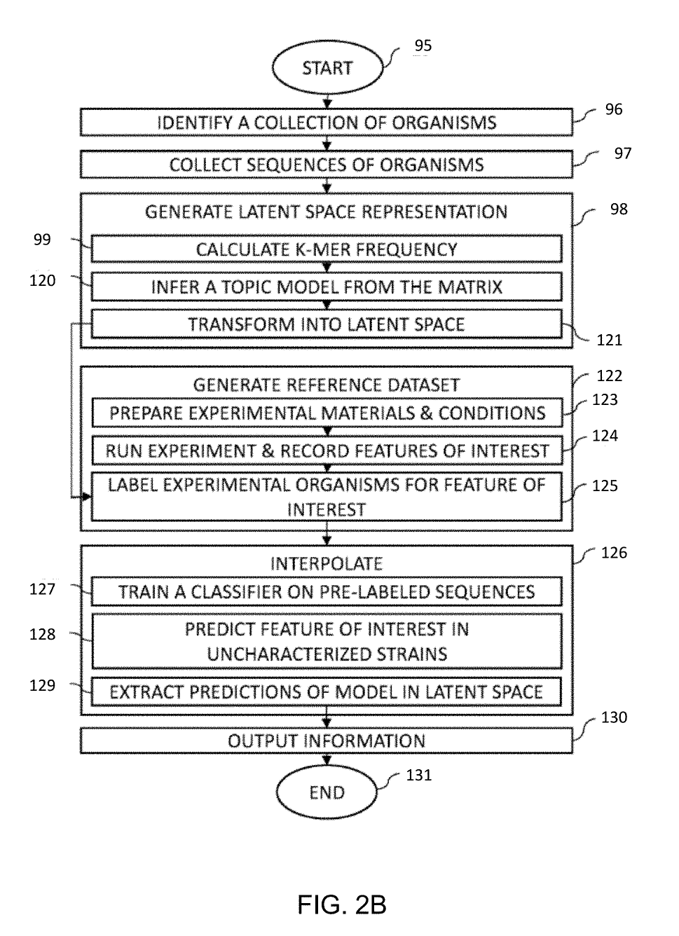

In contrast, the methods and systems disclose a different approach is to leverage marker gene sequences, 16S for bacteria and ITS for fungi, for studying relatedness rather than classification. These sequences do indicate broad evolutionary patterns and so capture a broad picture of phylogeny. In one embodiment, the technical problem of quickly identifying organisms that are related to an organism having a feature of interest can be achieved by using sequences, such as for example, marker gene sequences to create a representation of phylogeny. The system 5 can generate various phylogenetic representations such as clade diagrams or phylogenetic trees. An exemplary output is shown in FIG. 2A.

As shown in FIG. 2A, a phylogenetic tree representation 35 is shown for various organisms and their relationships to each other. These organisms are specific examples of organisms 10 which can be studied in a given environment 11. As shown, one of the organisms A4, have been observed experimentally to have a feature of interest, such as increased yield. Absent more information, knowing that organism A4 has genetic information that results in increased yield, does not permit any meaningful analysis to be performed relative to the other parents, siblings, and descendants shown in the tree.

As shown by the double headed arrows of the tree 35, organism A4, which has the increased yield feature, is related to organism A1, D2, E2, and F2. The relatedness of these organisms to organism A4 is determined by a latent space representation analysis. As discussed in more detail herein, each of these organisms have been sequenced and k-mers have been obtained for each organism. These in turn have been operated upon to generate a vector for each organism shown in the tree 35. Although the double headed arrows in the tree 35 do point out which organisms are related to organisms A4, the distance or angle of those arrows has no particular meaning.

When the vectors are represented in latent space representation 50, as shown in FIG. 1C, the relatedness of the other vectors to vector A4 is clear by virtue of the clustering in the vicinity of A4. The simplified two-dimensional model shown in FIG. 1C, is often a multi-dimensional vector spaces with a dimensionality greater than 3. As a result, the processing and measuring of distances or other similarity or relatedness metrics can be performed using various matrix representations. The vector lengths can be normalized to a common unit vector in some embodiments. Alternatively, the length of a given vector can be correlated with another feature of the organism or otherwise encode other data of interest.

From FIG. 1C, it is clear that transforming a phylogenetic tree, which presents an unwieldy and potentially unsolvable feature identification problem, can be overcome and solved, by generating a vector representation informed by a generative model such as an LDA model. The clustered vectors in the first quadrant are examples of target candidates that may be prioritized relative to the other vectors and thus facilitate a rapid identification of organisms that share the high yield feature of organisms A4.

Alternately, intensive individual study and characterization of individual organisms and the evolutionary patterns of their clade may be used to identify close relatives, their genomic composition, niches and lifestyle. While this is technically feasible, it requires considerable time and expense. As a result, it is not economically feasible for characterization of a large number of organisms. Therefore, the ability to be able to prioritize research and analysis by identifying target candidates using the system and techniques disclosed herein is a technical solution that solves the problem of finding beneficial organisms on a time scale that would otherwise be unmanageable.

The methods of the present disclosure are particularly useful for analysis of plant genomes. The genomes of plants 8, and particularly agriculturally relevant plants such as rice, soybean, cotton, corn and wheat, comprise several hundred million to several billion nucleotides. Given the large size of plant genomes it can be prohibitively expensive to conduct replicated experiments of one or more genomic, transcriptomic and/or proteomic analyses. The methods of the present disclosure, allow interpolation of an organism's features from sequences shorter than an entire genome, transcriptome or proteome. This facilitates rapid analysis and allows analysis and subsequent experimentation and testing of target candidates that have a dramatically increased likelihood of yielding useful results in terms of drought resistance, plant yield, and other parameters of interest.

In accordance with various embodiments, the present disclosure provides methods, compositions and apparatus for improving identification of one or more organisms' or one or more communities' phenotypes or functional characteristic and the genetic sequences or subsequence which identify the foregoing. Aspects of the exemplary embodiment relate to representing an organism 10 and its functional capabilities based on the occurrence of subsequences of a specified length, k-mers, within its genetic sequence. k-mers can be stored in database 25 or other electronic memory storage accessible by system 5. In turn, system 5 can in turn operate on training set data and apply classifiers and user inputs to direct various modeling routines such as generating a latent space representation and running various generative models and Gaussian processes. The system 5 can also output various reports that may include recommendations or the identity of target candidates, for instance via client device interface 30. The system can also receive queries and inputs to direct a model to focus on a particular feature such as a specific beneficial feature of an endophyte.

Techniques from Natural Language Processing and Topic Modeling, including Latent Dirichlet Allocation (LDA), can be used to analyze observed k-mer data from the organisms being analyzed to identify candidate targets. Specifically, LDA or another latent space model can be used to represent the k-mer content within each sequence. In one embodiment, a given latent space or similar space is discovered during training instead of constructed in advance of training a model such as a generative model. A natural representation of the k-mer content within each marker gene sequence can be achieved using a latent space approach using LDA or other machine learning models and techniques.

The LDA approach may be extended and transformed such that a sequence can be analogized to a document, which is constructed of topics. The topics can include words and by extension to a sequence, k-mers. These topics are shared across all sequences. The proportion of each topic in a strain's marker gene sequence is a natural metric for comparison. By examining topics, one or more embodiments calculate distances between strains based on correlated k-mer abundances. This can be undertaken rather than use the reads themselves. These distances can be used as relatedness metrics or scores. When used with a generative model, these distances can be used to identify target candidates that are related to organisms having known features of interest. In this way, if one endophyte is capable of providing a drought resistant trait to a plant, it is possible to identify related endophytes that are candidates for sharing similar phenotypes with respect to drought resistance.

In one embodiment, a sequence is a collection of topic proportions thereof. Further, in one embodiment a topic is a collection of k-mer frequencies. These collections are probability distributions. Unsupervised machine learning and these probability distributions and subsets thereof can be used with a generative model to output candidate targets. Such candidate targets are predicted to have a given feature of interest. These candidate targets can be used to direct further investigation and analysis to verify the presence of the expected feature of interest.

In one embodiment, a sequence comprises topics, and these topics are comprised of patterns of k-mers and correlations of k-mers. The topics are available for use across all sequences, though an individual sequences may lack one or more of the topics. The number of topics is chosen as the smallest number to completely represent the sequence data. In some embodiments, a fully generative model is built using a Dirichlet processor other machine learning generative model, allowing for any number of topics. However, some LDA implementations require setting the total number of topics to be used.

The number of non-null topics found will be less than or equal to the total number of topics that is set. The number of topics necessary to properly represent the sequence data can be found by varying the total number of topics, for example, by increasing the number of topics used until the number of non-null topics stabilizes. The proportion of each topic in an organism's sequence is a natural metric for comparison. By examining topics, the distances between strains based on correlated k-mer abundances, rather than the raw abundances themselves, were calculated. Some embodiments of the present disclosure provide a method for using sequences to create a representation of phylogeny such as for example a phylogenetic latent space representation or model.

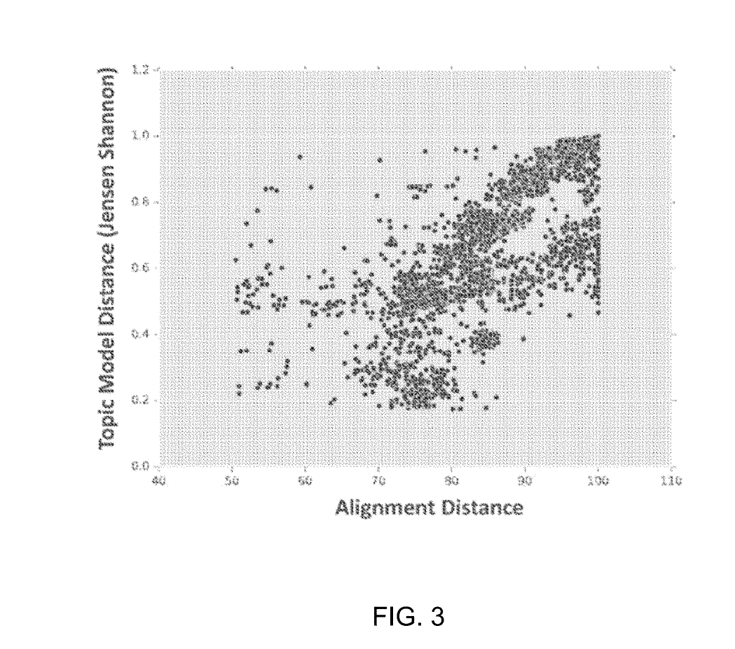

A distance metric, such as for example, a topic model distance, calculated on k-mer abundances by sequence recapitulates alignment distance (FIG. 3), while being considerably faster to calculate, and allowing comparisons from much further diverged sequences. From FIG. 3, the correlation between the distance metric disclosed herein and the corresponding alignment distance calculation validates the use of the distance metric. A given distance metric can be or can be correlated with a given probabilistic distance, such as Jensen-Shannon or others. Further, in one embodiment, the distance metric can be used as a relatedness measure, a relatedness score, or as a component of the foregoing.

In general, entropy, in the context of machine learning and data modeling, provides a metric to assess the amount of information content in a group of samples. A higher entropy value corresponds to more information content in the group. A lower entropy value indicates that the samples in a group are the same or closer to being the same. An entropy value of 0 indicates that all of the samples in a group are the same. Accordingly, low entropy groups of samples are not suitable for being training sets for a generative model.

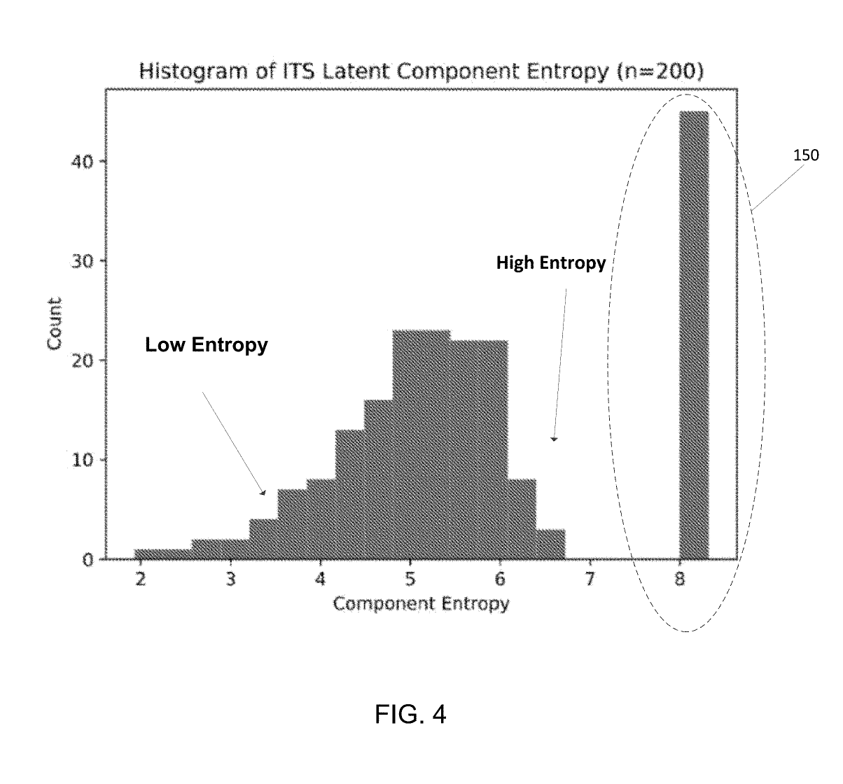

The entropy of topics comprised of k-mer frequencies can identify useful properties. For example, see the histogram of ITS (nuclear ribosomal internal transcribed spacer) latent space component entropy shown in FIG. 4. The wide range of entropy values is particularly noteworthy. The maximum entropy bin 150 corresponds to topics that were null, an artifact of the model fitting procedure. Low entropy k-mer topics can be used to identify specific groups, for example the fungal clade of Sordariomycetes. High entropy k-mer topics are frequently used and suitable for use in a training set. A group of samples of k-mers (corresponding to organisms or communities) having high entropy is suitable for use as a training set for a generative model.

As shown, in FIG. 4 and in general, high entropy k-mer topics represent subtle shifts in k-mer topic frequency, and may be used, for example to separate large clades such as the phyla Ascomycota and Basidiomycota. The wide range of entropy values is particularly noteworthy.

The latent representations of organism phylogeny generated from k-mer analysis of sequence can be used determine the properties of an unknown organism based on well described neighbors. Probabilistic similarity between organisms in the latent (topic) space may be defined using a suitable relatedness metric such as distance metric or a measure of divergence. In one embodiment, the relatedness metric is Jensen-Shannon divergence. It may be implicitly assumed that each sequence is a probability distribution over topics. Thus, for N sequences of observed organisms sequenced using system 3 of FIG. 1A, there are N different probability distributions.

Accordingly, for two sequences the distance between them is entropic, reflecting the property that a subsequence of one sequence would be found in another. This is a metric for considering whether the features of one organism are present in another.

Interpolation models, such as Gaussian Processes, may be used to determine whether an organism possesses features of interest and the probability of their occurrence. This may be achieved by identifying candidates for further confirmatory or investigatory experiments. Use of these latent spaces for interpolation of biological features provides a significant improvement in speed and accuracy of selection of organisms with desirable biological features from among the broad range of biological diversity, for example as may be found in a culture collection.

The process of the present disclosure may be implemented in any computer programming language, as a non-limiting example the process may be implemented in Python or R. Similarly, statistical methods and algorithms utilized in the present disclosure may be called from a variety of code libraries; a non-limiting example of a code library useful in the present disclosure is scikit learn (available at scikit-learn.org), which is useful for implementing the process of the present disclosure in Python.

Turning to FIG. 2A, an illustrative example of a process for predicting one or more organisms' or one or more communities' features and the sequence characteristics which identify these features. Although starting point 80 and ending point 95 are shown, these are merely exemplary references points, and other steps can precede and follow them without limitation.

The steps of the process of FIG. 2A are more fully described as follows. In one embodiment of the method, collecting sequences of organisms 82 is performed. This can be performed automatically or through user interactions. Sequences may be obtained from existing databases or isolated from samples of an organism or community. In some embodiments, the sequences are DNA sequences, RNA sequences, or protein sequences. In some embodiments, the sequences are marker gene sequences such as 16S, as may be found in the GreenGenes (available at greengenes.secondgenome.com) or SILVA databases (available at arb-silva.de) or sequenced from newly isolated bacteria; or ITS, as may be found in the UNITE (available at unite.ut.ee) or WARCUP (available at rdp.cme.msu.edu) databases or sequenced from newly isolated fungi.

In some embodiments, this step comprises an additional step of assigning identifiers to each organism, for example: randomly assigned identifiers of distinct individuals or communities, taxonomic classifications, morphological classifications, ecological classifications, geographic classifications, etc. In various embodiments, data is collected with regard to the organisms being sequenced using the organism data collection and modeling system shown and described with regard to FIG. 1A or other implementations thereof.

The method includes the step of generating a latent space representation 83 such as latent phylogenetic space. Where the collection of sequences in 82 comprises non-homologous sequences, the steps of 83 are performed separately for each set of homologous sequences. Sets of homologous sequences may be inferred using various methods, such as for example the clustering methods described in Weizhong Li, Limin Fu, Beifang Niu, Sitao Wu, John Wooley; Ultrafast clustering algorithms for metagenomic sequence analysis, Briefings in Bioinformatics, Volume 13, Issue 6, 1 Nov. 2012, Pages 656-668, available at doi.org/10.1093/bib/bbs035.

The method includes the step of calculating k-mer frequencies 84. The frequencies of all k-mers in the sequences are calculated, resulting in a matrix of organisms by k-mer frequencies. In some embodiments this step is repeated for k-mers of different lengths, and the processes in 83 and 87 run for each iteration of k. This may be done in order to select a k-mer size that maximizes predictivity and minimizes computational load. The smallest k-mer size that accurately represents phylogeny in the training data is chosen.

The method includes the step of determining a topic model from the matrix 85. A generative model is used to determine a topic model for the matrix of strains by k-mer frequencies. Any generative model may be used, including fully generative models built using a Dirichlet process prior, allowing for any number of topics or LDA implementations. Where it is necessary to set the number of topics a priori, for example for some LDA implementations, this step may be repeated with differing number of topics (generally increasing from a conservative starting number, for example 10) until the number of non-null topics stabilizes.

The method includes the step of transforming the topic model into latent space 86. Transform organisms of interest into this latent space using the transform function of the model. The result will be a matrix of organisms by latent vectors. In some embodiments, the result will be a matrix of strains by latent vectors, one for bacteria and one for fungi. In some embodiments, the result will be a matrix of a reference vector for a beneficial organism and a set of vectors for an organism to be compared therewith.

In some embodiments, the result will be generating a reference dataset 87. Determining organisms with features of interest 88 is performed. For a feature of interest, find all organisms that have that feature and its value (e.g. 1/0 for presence/absence or an integer for number of natural product pathways).

The method may include labeling all organisms for feature of interest 89. For organisms in the latent space (known sequence), append the feature value directly to the latent vector. This is shown in an illustrative example in FIG. 1C, wherein the vector for organism A4 is labeled “high yield feature.” For organisms without sequence data, the feature value may be bound to an identifier such as all genus-species matches in the latent space. This may give several values of a phenotype to the same organism in the latent space (repeated rows).

The method may include interpolating 90, which may include one or more of the following sub-steps. Further, the method may include training a classifier on pre-labeled sequences 91. The method may also include training a generative interpolation model, such as a Gaussian process model (regressor for continuous variables, classifier for binary ones), with the latent vectors as the independent variable (x) and the feature as the dependent variable (y) for all organisms with a feature (identified in 87). These methods can be implemented via libraries commonly known in the art such as scikit learn which is implemented in Python and available at scikit-learn.org. The method may also include predicting features of uncharacterized organisms 92.

Extracting the predictions of the model on all organisms in the latent space, for example, using the predict function of scikit 93 may also be performed. In addition, a list of candidate targets or other information can be output. Accordingly, the method includes the step of outputting information 94. For example, these predictions may be used as candidate properties. These can be recommended for target candidates. In general, the foregoing and other methods can be implemented on system 5 as part of the overall modeling and data collection platform 3 of FIG. 1A.

The methods of the present disclosure can be coupled with an experimental pipeline, screening assay or other means of testing organisms so that the results of the testing allow further analysis and target candidate identification which organismal features are driving the results of the tests without necessarily profiling, sequencing, and deeply studying each organism (FIG. 2B). Given a known feature of interest, for example as learned from an assay, organisms possessing the desired feature can be selected and prioritized for study. Features of interest can either be identified from machine learning, retrospectively looking at the commonalities between which organisms performed highly, or prospectively, based on scientific hypotheses.

Turning to FIG. 2B, an illustrative example of a process for predicting one or more organism's or one or more communities' features as determined by an experimental assay and the sequence characteristics which identify these phenotypes. Although starting point 80 and ending point 95 are shown, these are merely exemplary references points, and other steps can precede and follow them without limitation.

The steps of the process of FIG. 2B are more fully described as follows. The method may include identifying a collection of organisms 96. Organisms or communities of organisms are identified, and in some embodiments assigned identifiers, for example: randomly assigned identifiers of distinct individuals or communities, taxonomic classifications, morphological classifications, ecological classifications, geographic classifications, etc. This step is optional. Other steps of any method disclosed herein may be optional. In some embodiments this step is combined with step 97. In various embodiments, data is collected with regard to the organisms using the organism data collection and modeling system shown and described with regard to FIG. 1A or other implementations thereof.

The method may include collecting sequences of organisms 97. Sequences may be obtained from existing databases or isolated from samples of the organism or community. In some embodiments, the sequences are DNA sequences, RNA sequences, or protein sequences.

The method may include generating latent phylogenetic space 98. Where the collection of sequences in 97 comprises non-homologous sequences, the steps of 98 are performed separately for each set of homologous sequences. The method may include calculating k-mer frequencies 99. The frequency of all k-mers in the sequences is calculated, resulting in a matrix of strains by k-mer frequencies. In some embodiments this step is repeated for k-mers of different lengths, the processes in 98 and 122 run for each iteration of k, in order to inform selection of k for a particular sequence set and application.

The method may include determining a topic model 120. Using a generative model, such as LDA for example using the scikit learn implementation, to determine a topic model for matrix of strains by k-mer frequencies. This step may be repeated using differing numbers of topics. The method may include transforming into latent space 121. The topic model is transformed into a matrix of strains by latent vectors, for example using the transform function of the LDA model as implemented in scikit learn. The result is a matrix of strains by latent vectors. The method may include generating a reference dataset 122. A variety of observed or measured features or phenotypes of an organism may be used.

Further, the method may include preparing experimental materials and conditions 123. In the case of observations of an organism in natural conditions, preparation of experimental materials may include selection of a geographic region, such as a field site, or identification of study participants. It may also include selection and preparation of equipment necessary to capture observations and measurements. In highly controlled experimental conditions this step may include selection or sterilization of equipment, development of modified experimental organisms, etc.

The method may include running experiment and recording features of interest 124. For a phenotype of interest, find all strains that have that phenotype and its value. Features recorded may be represented by binary, categorical or continuous variables. The method may include labeling experimental organisms for feature of interest 125. For organisms in the latent space (known sequence), append the recorded phenotype directly to the latent vector. For organisms without sequence data, the recorded phenotype may be bound to all identifier matches in the latent space.

The method may include interpolating 126, which may include one or more of the following substeps. The method may include training a classifier on pre-labeled sequences 127. Train a generative interpolation model, such as a Gaussian process model (regressor for continuous variables, classifier for binary ones) with the latent vectors as the independent variable (x) and the phenotype as the dependent variable (y) for all strains with a phenotype (generated in 122). The method may include predicting features of uncharacterized strains 128. The method may include extracting the predictions of the model on all strains in the latent space 129. The method may include outputting information 130. These predictions may be may be used to prioritize organisms for experimentation.

### EXAMPLES

### Example 1. Prediction of Beneficial Endophytes from Marker Gene Sequences

Generation of a reference dataset using domain annotation of reference genome sequences. Using publicly available whole genome sequences, the number of biosynthetic gene clusters (PKS, NRPS) was calculated by counting the number of polydomain proteins, as defined by Pfam, containing ketoacyl synthase domains ‘ketoacyl-sync’), or acyltransferase domains (‘Acyl_transf_1’), or all three of ‘PP-binding’, ‘Condensation’, and ‘AMP-binding’ in one protein. In well-assembled genomes, it has been shown that the first two are a good estimate of the number of biosynthetic gene clusters. The third rule (counting all three domains PP-binding, Condensation, and AMP-binding) was included as assembly errors on biosynthetic gene clusters, due to repeated subsequences, tend to produce estimated proteins with numerous domains of all three of these types, obscuring the final synthase or transferase domain. All proteins can at most contribute once to this count: the presence of a ketoacyl synthase and an acyl transferase domain only counts as 1 predicted gene cluster.

Generate Latent Phylogenetic Space.

A set of 16S nucleotide sequences was collected for bacteria from the GreenGenes and SILVA databases and ITS nucleotide sequences for fungi from the WARCUP and UNITE ITS databases. For each set of sequences separately, the frequency of all 6-mers in the sequences was calculated, then used the scikit learn LDA implementation to determine a topic model for the resulting matrix of strains by 6-mer frequencies. For bacteria 800 topics were used, for fungi 200 topics were used. Strains of interest were then transformed into this latent space using the transform function of the LDA model. The result was a matrix of strains by latent vectors, one for bacteria and one for fungi.

Using the taxonomy of the whole genome sequenced organisms, the predicted number of biosynthetic gene clusters were matched to individual strains in public databases and the previously generated latent spaces.

Interpolation.

A Gaussian process model was then trained with an Exponential/Matern Kernel (nu=0.5), using scikit-learn, separately on latent spaces based on the 16S and the ITS sequences. The number of biosynthetic gene clusters was predicted for whole genome sequenced but uncharacterized strains. The number of predicted number of gene clusters differed from the state of the art estimate using the program PRISM for these strains by +/−1 out of 26, while performing the estimation for hundreds of strains in seconds. The estimation with PRISM took approximately 12 hours per strain. Additionally, PRISM could only be used on whole genome sequenced strains, whereas the number of biosynthetic gene clusters for all strains having a marker gene sequence could be predicted.

Strain taxonomy was predicted using a nearest neighbor classifier (n=5), as a naïve, discrete form of interpolation for the GreenGenes strains (Bacteria) and the UNITE strains (Fungi), using the latent space mentioned above. Scikit-learn's nearest neighbor classifier with n=5 on the public data was used. Accuracy was achieved at 94% at the genus level for bacteria and 96.5% for fungi. At the family level, where taxonomic assignment is more trustworthy, especially in fungi, accuracy of >97% was achieved for both. At higher levels, the results were nearly perfect.

### Example 2. Prediction of Indole-3-Acetamide Hydrolase Gene Frequency from Marker Gene Sequences

Generation of a Reference Dataset Using Hidden Markov Model (HM114) for Gene of Interest.

Protein sequences annotated as indole-3-acetamide hydrolase (IaaH) were downloaded from NCBI's non-redundant protein database. An experimentally validated IaaH protein sequence was chosen as the seed and jackhmmer (available at hmmer.org/) was run to select a homologous reference set and generate a HMM. This HMM was used to determine the frequency of IaaH genes in the sets of translated coding sequences from publicly available whole genome sequences. This process was run separately for bacterial IaaH reference sequences and genomes and fungal IaaH reference sequences and fungal genomes.

Generate Latent Phylogenetic Space.

Latent space representations were generated of 16S nucleotide sequences from obtained from the GreenGenes and SILVA databases for bacteria, using 200 topics; and the WARCUP and UNITE ITS databases and other sequenced fungi, using 200 topics. Using the taxonomy of the whole genome sequenced organisms, the predicted number of IaaH was matched to the individual strains and appended to the latent vector.

Interpolation.

A Gaussian process model was then trained with an Exponential/Matern Kernel (nu=0.5), using scikit-learn, separately on latent spaces based on 16S and ITS nucleotide sequences for bacteria and fungi. The number of IaaH gene clusters was predicted for whole genome sequenced but uncharacterized strains. For validation, the predicted number of IaaH gene clusters was then compared with the known number IaaH gene clusters in those organisms

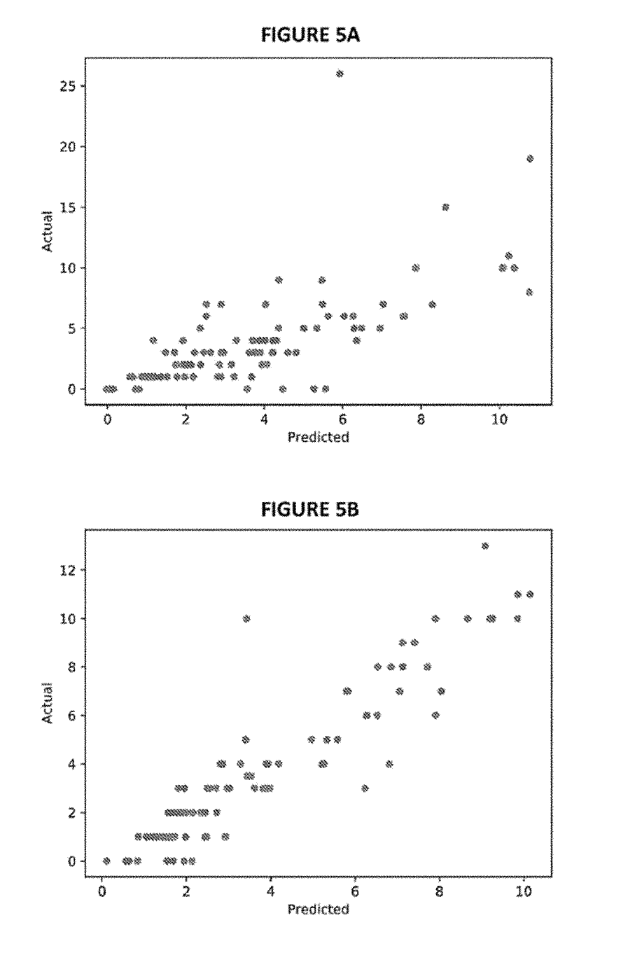

FIG. 5A shows the strong correspondence between the actual number of IaaH gene clusters in genomes of uncharacterized bacterial strains (as shown on the y-axis) and the predicted number of IaaH gene clusters predicted for those strains (as shown on the x-axis).

FIG. 5B shows strong correspondence between the actual number of IaaH gene clusters in genomes of uncharacterized fungal strains on the (y-axis) and the predicted number of IaaH gene clusters predicted for those strains.

### Example 3. Prediction of Plant Growth Promotion Phenotype from Marker Gene Sequences

Among other applications, the methods of the present disclosure may be applied to phenotypic characterization of plants or animals exposed to one or more organisms, for example bacteria or fungi. The methods can be integrated into a preliminary screening assay and may be used to prioritize treatments, for example bacterial or fungal treatments, for more focused, and more expensive, secondary assays. The following is a representative, non-limiting, example of methods which may be used to characterize the ability of a bacterium or a fungus to affect a growth of a variety of plants treated with the bacterium or fungus and identification of features of these bacteria or fungi that are driving the plant growth phenotypes.

Isolation of Bacteria and Fungi.

Bacterial and fungal strains to be screened for plant growth promoting activity may be isolated from various environments 11, such as shown in FIG. 1A. These may include natural occurring or artificial abiotic or biotic environments, including from within surface sterilized wild or domesticated plants. The individual microbes or communities of microbes may be selected for isolation and cultivation as part of follow on experiments using the systems and methods disclosed herein. Isolated microbes may be assigned strain identifiers, for example: randomly assigned identifiers of unique isolates, taxonomic classifications, morphological classifications, etc.

Sequencing of Isolated Bacteria or Fungi.

Nucleic acids of the microbes are extracted by, and characterized by the nucleic acid sequences, for example using primer sequences such as those listed in Table 1, or whole genome sequencing, or metagenome sequencing.

Generate Latent Phylogenetic Space.

For each set of homologous genomic sequences generated, the frequency of all k-mers in the sequences is calculated, then a generative model such as LDA is used to determine a topic model for the matrix of microbes by k-mer frequencies, the topic model is then transformed into a matrix of strains by latent vectors, one matrix per homologous genomic sequence.

TABLE 1
Examples of marker gene primer sequences useful in identifting microbes
of the present disclosure

| Primers | Genomic locus |
|---|---|
| 27f (5′-AGAGTTTGATYMTGGCTCAG-3′) (SEQ ID NO: 1) | 16S |
| 1492r (5′-GGTTACCTTGTTACGACTT-3′) (SEQ ID NO: 2) | |
| 515f (5′-GTGYCAGCMGCCGCGGTAA-3′) (SEQ ID NO: 3) | 16S |
| 806r (5′-GGACTACNVGGGTWTCTAAT-3′) (SEQ ID NO: 4) | |
| ITS_1 (5′-CTTGGTCATTTAGAGGAAGTAA-3′) (SEQ ID NO: 5) | ITS |
| LR5 (5′-TCCTGAGGGAAACTTCG-3′) (SEQ ID NO: 8) | |
| ITS_2 (5′-GCTGCGTTCTTCATCGATGC-3′) (SEQ ID NO: 6) | ITS |
| ITS_3 (5′-GCATCGATGAAGAACGCAGC-3′) (SEQ ID NO: 7) | |
| PGK (5′-GTYGAYTTCAAYGTYCC-3′) (SEQ ID NO: 9) | phosphoglycerate |
| PGK (5′-ACACCDGGDGGRCCGTTCCA-3′) (SEQ ID NO: 10) | kinase |
| ACT512f, Actin, primer-amplicon F (5′-ATGTGCAAGGCCGGTTTCG- | actin |
| 3′) (SEQ ID NO: 11) | |
| ACT783r, Actin, primer-amplicon R (5′-TACGAGTCCTTCTGGCCCAT- | |
| 3′) (SEQ ID NO: 12) | |
| fusA-f2, elongation factor G, primer-amplicon F (5′- | elongation factor |
| TCGCGTTCGTTAACAAAATGGACCGTAT-3′) (SEQ ID NO: 13) | G |
| fusA-R2, elongation factor G, primer-amplicon R (5′- | |
| TCGCCAGACGGCCCAGAGCCAGACCCAT-3′) (SEQ ID NO: 14) | |
| RPB1-Af, largest subunit of RNA polymerase II, primer- | largest subunit of |
| amplicon F (5′-GARTGYCCDGGDCAYTTYGG-3′) (SEQ ID NO: 15) | RNA polymerase |
| RPB1-Cr, largest subunit of RNA polymerase II, primer- | II |
| amplicon R (5′-CCNGCDATNTCRTTRTCCATRTA-3′) (SEQ ID NO: 16) | |
| LR0R, long subunit rRNA gene, primer-amplicon F (5′- | long subunit |
| ACCCGCTGAACTTAAGC-3′) (SEQ ID NO: 17) | rRNA gene |
| LR5, long subunit rRNA gene, primer-amplicon R (5′- | |
| TCCTGAGGGAAACTTCG-3′) (SEQ ID NO: 8) | |
| bRPB2-7.1R, second largest subunit of RNA polymerase II, | second largest |
| primer-amplicon R (5′-CCCATRGCYTGYTTMCCCATDGC-3′) (SEQ | subunit of RNA |
| ID NO: 18) | polymerase II |
| fRPB2-5F, second largest subunit of RNA polymerase II, | |
| primer-amplicon F (5′-GAYGAYMGWGATCAYTTYGG-3′) (SEQ | |
| ID NO: 19) | |
| NS1 (5′-GTAGTCATATGCTTGTCTC-3′) (SEQ ID NO: 20) | SSU, small |
| NS4 (5′-CTTCCGTCAATTCCTTTAAG-3′) (SEQ ID NO: 21) | subunit rRNA |
| | gene |
| SR1R (5′-TACCTGGTTGATTCTGCCAGT-3′) (SEQ ID NO: 22) | SSU, small |
| NS4 (5′-CTTCCGTCAATTCCTTTAAG-3′) (SEQ ID NO: 21) | subunit rRNA |
| | gene |
| Btub2Fd, beta-tubulin, primer-amplicon F (5′- | Beta-tubulin |
| GTBCACCTYCARACCGGYCARTG-3′) (SEQ ID NO: 23) | |
| Btub4Rd, beta-tubulin, primer-amplicon R (5′- | |
| CCRGAYTGRCCRAARACRAAGTTGTC-3′) (SEQ ID NO: 24) | |

### Generation of Reference Data Set by Preliminary Screening of Microbial Treatments for Plant Growth Promotion

The set of microbes represented in the latent space representation generated above may be screened for a feature such germination, root area, root length, and or shoot length. Exemplary high throughput assays suitable for a number of crops follow. One or more of these assays may be performed before, after or concurrent with generation of the latent space representation. A feature may be a continuous or categorical variable based on results of one or more assays in one or more crops.

Assay of Soy Seedling Vigor

Seed Preparation:

The lot quality of soybean seeds is first assessed by testing germination of 100 seeds. Seeds are placed, 8 seeds per petri dish, on filter paper in petri dishes, 12 mL of water is added to each plate and plates are incubated for 3 days at 24° C. The process should be repeated with a fresh seed lot if fewer than 95% of the seeds have germinated. One thousand soybean seeds are then surface sterilized by co-incubation with chlorine gas in a 20×30 cm container placed in a chemical fume hood for 16 hours. Percent germination of 50 seeds, per sterilization batch, is tested as above and confirmed to be greater than 95%.

Preparation of Endophyte Treatments:

Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10̂6 spores/ml utilizing water. 3 μl of spore suspension is used per soy seed (˜10̂3 CFUs/seed is obtained). Control treatments are prepared by adding equivalent volumes of sterile water to seeds.

Assay of Seedling Vigor:

Two rolled pieces of germination paper are placed in a sterile glass gar with 50 mL sterile water, then removed when completely saturated. Next, the papers are separated and inoculated seeds are placed at approximately 1 cm intervals along the length of one sheet of moistened germination paper, at least 2.5 cm from the top of the paper and 3.8 cm from the edge of the paper. The second sheet of is placed on top of the soy seeds and the layered papers and seeds are loosely rolled into a tube.

Each tube is secured with a rubber band around the middle and placed in a single sterile glass jar and covered loosely with a lid. For each treatment, three jars with 15 seeds per jar are prepared. The position of jars within the growth chamber is randomized. Jars are incubated at 60% relative humidity, and 22° C. day, 18° C. night with 12 hours light and 12 hours dark for 4 days and then the lids are removed and the jars incubated for an additional 7 days. Then the germinated soy seedlings are weighed and photographed and root length and root surface area are scored as follows.

Dirt, excess water, seed coats and other debris is removed from seedlings to allow accurate scanning of the roots. Individual seedlings are laid out on clear plastic trays and trays are arranged on an Epson Expression 11000XL scanner (Epson America, Inc., Long Beach Calif.). Roots are manually arranged to reduce the amount of overlap. For root measurements, shoots are removed if the shape of the shoot causes it to overlap the roots.

The WinRHIZO software version Arabidopsis Pro2016a (Regents Instruments, Quebec Canada) is used with the following acquisition settings: greyscale 4000 dpi image, speed priority, overlapping (1 object), Root Morphology: Precision (standard), Crossing Detection (normal). The scanning area is set to the maximum scanner area. When the scan is completed, the root area is selected and root length and root surface area are measured.

Statistical analysis is performed using R (R Core Team, 2016. R: A language and environment for statistical computing. R Foundation for Statistical Computing, Vienna, Austria. R-project.org/) or a similar statistical software program.

Assay of Corn Seedling Vigor

Seed Preparation:

The lot quality of corn seeds is first evaluated for germination by transfer of 100 seeds and with 3.5 mL of water to a filter paper lined petri dish. Seeds are incubated for 3 days at 24° C. The process should be repeated with a fresh seed lot if fewer than 95% of the seeds have germinated. One thousand corn seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional Reagent Preparation:

7.5% PEG 6000 (Calbiochem, San Diego, Calif.) is prepared by adding 75 g of PEG to 1000 mL of water, then stirred on a warm hot plate until the PEG is fully dissolved. The solution is then autoclaved.

Preparation of Endophyte Treatments:

Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10̂6 spores/ml utilizing water. 3 μl of spore suspension is used per corn seed (˜10̂3 CFUs/seed is obtained). Control treatments are prepared by adding equivalent volumes of sterile water to seeds.

Assay of seedling vigor: Either 25 ml of sterile water or, optionally, 25 ml of PEG solution as prepared above, is added to each Cyg™ germination pouch (Mega International, Newport, Minn.) and place into pouch rack (Mega International, Newport, Minn.). Sterile forceps are used to place corn seeds prepared as above into every other perforation in the germination pouch. Seeds are fitted snugly into each perforation to ensure they do not shift when moving the pouches. Before and in between treatments forceps are sterilized using ethanol and flame and workspace wiped down with 70% ethanol. For each treatment, three pouches with 15 seeds per pouch are prepared.

Next, the germination racks with germination pouches are placed into plastic tubs, and covered with perforated plastic wrap to prevent drying. Tubs are incubated at 60% relative humidity, and 22° C. day, 18° C. night with 12 hours light and 12 hours dark for 6 days to allow for germination and root length growth. Placement of pouches within racks and racks/tubs within the growth chamber is randomized to minimize positional effect. At the end of 6 days the corn seeds are scored manually for germination, root and shoot length.

Statistical analysis is performed using R or a similar statistical software program.

Assay of Wheat Seedling Vigor

Seed Preparation:

The lot of wheat seeds is first evaluated for germination by transfer of 100 seeds and with 8 mL of water to a filter paper lined petri dish. Seeds are incubated for 3 days at 24° C. The process should be repeated with a fresh seed lot if fewer than 95% of the seeds have germinated. Wheat seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional Reagent Preparation:

7.5% polyethylene glycol (PEG) is prepared by adding 75 g of PEG to 1000 mL of water, then stirring on a warm hot plate until the PEG is fully dissolved. The solution is then autoclaved.

Preparation of Endophyte Treatments:

Spore solutions are made by rinsing and scraping spores from agar slants which have been growing for about 1 month. Rinsing was done with 0.05% Silwet. Solutions are passed through Miracloth to filter out mycelia. Spores per ml are counted under a microscope using a hemocytometer. The stock suspension is then diluted into 10̂6 spores/ml utilizing water. 3 μl of spore suspension is used per wheat seed (˜10̂3 CFUs/seed was obtained). Seeds and spores are combined a 50 mL falcon tube and gently shaken for 5-10 seconds until thoroughly coated. Control treatments are prepared by adding equivalent volumes of sterile water to seeds.

Assay of Seedling Vigor:

Petri dishes are prepared by adding four sheets of sterile heavy weight seed germination paper, then adding either 50 mL of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface.

Along the edge of the plate across from the covered side wall, 15 inoculated wheat seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then wheat seeds scored manually for germination, root and shoot length.

In one embodiment, statistical analysis is performed using R or a similar statistical software program.

Merge Latent Space Representations with Assay Results.

Strain identifiers are used to match the results of the assays, e.g. the plant phenotypes of plants treated with the microbe, with latent space as generated above. Accordingly, by reviewing the latent space as combined with the foregoing results, the clustering or relatedness of the vectors in the latent space provide guidance that shows how the results of the assay map to the organisms that are identified as related based on the proximity of one vector to another or another distance metric applicable to the latent space representation.

Interpolation.

A generative interpolation model, such as a Gaussian process model (a regressor for continuous variables, a classifier for binary variables), is then trained with the latent vectors as the independent variable (x) and the phenotype as the dependent variable (y) for all microbes which were used as plant treatments in an assay. The analysis performed can be used to generate or analyze or confirm correlations and indications of relatedness between one or more organisms.

Predict Features of Uncharacterized Strains:

The model is then used to predict the plant phenotypic effecting properties of the uncharacterized microbes. These predictions may be used to prioritize strains for further testing. Further testing may comprise testing the assays described above and or other assays such as field trials, greenhouse experiments, or molecular characterization.

### Other Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, as noted above, the described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus or system for performing the operations herein. Such an apparatus or system may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may include information resulting from a computing process, where the information is stored on a non-transitory, computer readable storage medium and may include any embodiment of a computer program product or other data described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
training a generative topic model on a set of training crop sequence information, the generative topic model configured to convert crop sequence information into a latent space representation of k-mers within the crop sequence information;
accessing first crop sequence information representative of each of a first set of crops that include a crop feature;
generating a latent space representation of k-mers within the first crop sequence information representative of the first set of crops using the generative topic model;
training a generative interpolation model using the generated latent space representation, the generative interpolation model configured to classify crop sequence information representative of a target crop to determine a likelihood that the target crop includes the crop feature; and
in response to receiving a request from a requesting entity via a client device for a recommendation for a crop that includes the crop feature:
accessing second crop sequence information representative of each of a second set of crops;
identifying a subset of the second set of crops that include the crop feature by applying the generative interpolation model to the second crop sequence information, each crop within the identified subset of crops corresponding to a probability produced by the generative interpolation model that the crop includes the crop feature; and
modifying an interface of the client device to include information identifying each crop of the subset of crops and identifying, for each identified crop, the corresponding probability that the crop includes the crop feature.

2. The method of claim 1, wherein the received request is produced by the client device in response to the requesting entity identifying the crop feature via an interface element displayed by the client device.

3. The method of claim 1, wherein the crop feature comprises an above-threshold expected crop performance.

4. The method of claim 3, wherein modifying the interface of the client device further comprises identifying, for each identified crop, a measure of expected crop performance, the measure of expected crop performance determined based on the probability that the identified crop includes the crop feature.

5. The method of claim 1, further comprising:
receiving information associated with one or more of the identified subset of crops, the received information indicating whether or not each of the one or more of the identified subset of crops includes the crop feature;
generating an updated latent space representation based on the received information; and
re-training the generative interpolation model using the generated updated latent space representation.

6. The method of claim 5, further comprising:
in response to receiving a second request from the requesting entity via the client device for a second recommendation for a second crop that includes the crop feature:
identifying a second subset of the second set of crops that include the crop feature by applying the re-trained generative interpolation model to the second crop sequence information; and
modifying the interface of the client device to include information identifying each crop of the second subset of crops.

7. The method of claim 1, wherein the requesting entity comprises one or more of a crop grower, a crop broker, or an agronomist.

8. The method of claim 1, wherein the latent space representation comprises a matrix, and wherein each column of the matrix corresponds to a presence of one or more k-mers within a crop sequence.

9. The method of claim 1, wherein the interface comprises an interface element that enables the requesting entity to select a crop type, and wherein each of the second set of crops is associated with the selected crop type.

10. The method of claim 1, wherein the identified subset of the second set of crops comprises each crop of the second set of crops that corresponds to an above-threshold probability that the crop includes the crop feature.

11. A method comprising:
accessing first genetic sequence information representative of each of a first set of organisms that include an organism feature;
generating a latent space representation of k-mers within the first genetic sequence information representative of the first set of organisms;
training a generative interpolation model using the generated latent space representation, the generative interpolation model configured to classify genetic sequence information representative of a target organism to determine whether the target organism includes the organism feature;
accessing second genetic sequence information representative of each of a second set of organisms; and
identifying a subset of the second set of organisms that include the organism feature by applying the generative interpolation model to the second genetic sequence information.

12. The method of claim 11, wherein the latent space representation is generated using a generative topic model.

13. The method of claim 12, wherein the generative topic model comprises a latent Dirichlet allocation model.

14. The method of claim 12, wherein the generative topic model is trained on a set of training genetic sequence information.

15. The method of claim 14, wherein the set of training genetic sequence information comprises a first subset of training genetic sequence information representative of organisms that include the organism feature and a second subset of training genetic sequence information representative of organisms that do not include the organism feature.

16. The method of claim 12, wherein the generative topic model is periodically updated in response to receiving additional genetic sequence information for inclusion in the set of training genetic sequence information.

17. The method of claim 12, wherein the generative topic model is re-trained in response to receiving feedback indicating a predictiveness of one or more of k-mers for the organism feature.

18. The method of claim 11, wherein the latent space representation is re-generated in response to a triggering event, and wherein the re-generated latent space representation includes at least one input variable not included in the latent space representation.

19. The method of claim 18, wherein the triggering event comprises one or more of: a passage of a threshold interval of time, determining that a threshold portion of the identified subset of organisms does not include the organism feature, and obtaining additional genetic sequence information for use in generating the latent space representation.

20. The method of claim 11, wherein the latent space representation comprises a matrix, wherein each row of the matrix corresponds to an organism of the first set of organisms, and wherein each column of the matrix corresponds to a presence of one or more k-mers within genetic sequence information.

21. The method of claim 11, wherein the latent space representation comprises a sparse representation of k-mers within the first genetic sequence information.

22. The method of claim 11, wherein the first genetic sequence information comprises one or more of: DNA sequence information, RNA sequence information, whole genome sequence information, marker gene sequence information, and amino acid sequence information.

23. The method of claim 11, wherein the generative interpolation model is trained based in part on a distance between input variables in the latent space representation.

24. The method of claim 11, wherein applying the generative interpolation model to the second genetic sequence information comprises:
converting the second genetic sequence information into a second latent space representation, wherein each column of the latent space representation is associated with a same variable as a corresponding column in the second latent space representation; and
determining a likelihood that each organism in the second set of organisms includes the organism feature based on, for each variable of the latent space representation, a covariance between a weighted average value of the variable in the latent space representation and a value of the variable in the second latent space representation.

25. The method of claim 11, wherein the generative interpolation model comprises a Gaussian process model.

26. The method of claim 11, wherein classifying genetic sequence information representative of the target organism comprises determining a likelihood that the target organism includes the organism feature.

27. The method of claim 26, wherein each organism in the identified subset of organisms is associated with an above-threshold likelihood that the organism includes the organism feature.

28. The method of claim 11, wherein the organisms of the first set of organisms and the second set of organisms comprise one of: bacteria, archaebacteria, eubacteria, protista, fungi, plantae, and animalia.

29. The method of claim 11, wherein the organisms of the first set of organisms and the second set of organisms are plants.

30. The method of claim 29, wherein the plants are monocots.

31. The method of claim 29, wherein the plants are dicots.

32. The method of claim 29, wherein the plants comprise one or more of: corn, soybeans, cotton, wheat, rice, barley, oats, tomatoes, canola, and sorghum.

33. The method of claim 29, wherein the plants comprise different genotypes of a particular type of crop.

34. The method of claim 11, wherein the first set of organisms comprises one of: at least 10 different organisms, at least 50 different organisms, at least 100 different organisms, at least 500 different organisms, at least 1000 different organisms, at least 10 different organism communities, at least 50 different organism communities, at least 100 different organism communities, at least 500 different organism communities, and at least 1000 different organism communities.

35. The method of claim 11, wherein the identified subset of the second set of organisms includes one or more communities of organisms.

36. The method of claim 11, wherein the identified subset of the second set of organisms includes multiple different types of crops.

37. The method of claim 21, wherein the identified subset of the second set of organisms includes different genotypes of a particular type of crop.

38. The method of claim 11, wherein the organism feature comprises one or more of: a genomic composition, a frequency of biosynthetic gene clusters, a taxonomic categorization, a morphology, an environmental niche, a lifestyle, a resistance to desiccation, a spore formation, a suitability for manufacturing or harvesting, a viability, a compatibility with commercial practices, a stability in viability over time or ranges of environmental conditions, a compatibility with select formulations and chemical preparations, a chemical diversity production, a metabolite production, a pathogenicity, a toxicity, a metagenomic composition, a frequency of genes, a yield associated with an organism, a yield increase associated with an organism, and a crop performance.

39. The method of claim 11, further comprising prioritizing the testing of the identified subset of organisms in an experiment to determine if tested organisms include the organism feature.

40. The method of claim 11, wherein the second genetic sequence information is accessed in response to a request for a recommendation for the subset of the second set of organisms received from a requesting entity via a client device.

41. The method of claim 40, wherein identifying a subset of the second set of organisms comprises modifying an interface displayed by the client device to include information representative of the subset of the second set of organisms.

42. The method of claim 41, wherein the interface is further modified to display, for each identified organism in the subset of the second set of organisms, a corresponding representation of a likelihood that the identified organism includes the organism feature.

43. The method of claim 40, wherein the second genetic sequence information is received from a device or data storage entity associated with the requesting entity.

44. The method of claim 40, wherein the second set of organisms comprise organisms selected by the requesting entity.

45. The method of claim 11, wherein the first set of organisms and the second set of organisms comprise microbes or communities of microbes.

46. The method of claim 45, wherein identifying the subset of the second set of organisms comprises modifying an interface of a client device to display, for each organism of the identified subset of the second set of organisms, a recommendation to apply a microbe or community of microbes as a crop treatment.

47. A non-transitory computer-readable storage medium storing executable computer instructions that, when executed by one or more processors, cause the processors to perform steps comprising:
accessing first genetic sequence information representative of each of a first set of organisms that include an organism feature;
generating a latent space representation of k-mers within the first genetic sequence information representative of the first set of organisms;
training a generative interpolation model using the generated latent space representation, the generative interpolation model configured to classify genetic sequence information representative of a target organism to determine whether the target organism includes the organism feature;
accessing second genetic sequence information representative of each of a second set of organisms; and
identifying a subset of the second set of organisms that include the organism feature by applying the generative interpolation model to the second genetic sequence information.
